(19) United States
(12) Patent Application Publication
(10) Pub. No.: US 2024/0093028 A1
(43) Pub. Date: Mar. 21, 2024

(54) Thermoplastic Elastomer De-Tackifier Additive and Compounds

(71) Applicant: Scott M. Epstein, Medfield, MA (US)

(72) Inventor: Scott M. Epstein, Medfield, MA (US)

(21) Appl. No.: 18/467,784

(22) Filed: Sep. 15, 2023

Related U.S. Application Data
(60) Provisional application No. 63/375,787, filed on Sep. 15, 2022.

(51) Int. Cl. C08L 75/06; A61K 9/16

(52) U.S. Cl. CPC C08L 75/06; A61K 9/1641; A61K 9/1682

(57) ABSTRACT

A material includes a thermoplastic elastomer and a polymer additive substantially uniformly distributed within the material. The polymer additive has hydrophilic segments and hydrophobic segments. The concentration of the polymer additive in the material is less than or equal to about 40% by volume and greater than or equal to about 1% by volume.

### CROSS-REFERENCE TO RELATED APPLICATIONS

This application claims priority to U.S. Provisional Application No. 63/375,787, titled “Thermoplastic Elastomer De-Tackifier Additive and Compounds,” filed on Sep. 15, 2022, which is hereby incorporated by reference.

### TECHNICAL FIELD

This application relates generally to thermoplastic elastomers.

### BACKGROUND

Thermoplastic elastomers (TPEs), specifically soft-durometer polymer resins and more specifically thermoplastic polyurethanes (TPUs), when converted using thermoplastic processing, when solvated and cast, or when used as a coating have a tacky sticky surface. This undesirable feature is typically diminished by secondary processing including lubricous or higher durometer, lower surface-energy coatings, adding cost and additional handing requirements.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a material comprising a thermoplastic elastomer; and a polymer additive substantially uniformly distributed within the material, the polymer additive having hydrophilic segments and hydrophobic segments, wherein a concentration of the polymer additive in the material is less than or equal to about 40% by volume and greater than or equal to about 1% by volume (e.g., about 1% to about 40% by volume). In one or more embodiments, the thermoplastic elastomer comprises a thermoplastic polyurethane. In one or more embodiments, the polymer additive comprises a partially hydrolyzed polyacrylonitrile. In one or more embodiments, the partially hydrolyzed polyacrylonitrile has a hydrolysis percentage of greater than equal to about 30% and less than or equal to about 50% (e.g., about 30% to about 50%).

In one or more embodiments, the material comprises a plurality of pellets, each pellet having substantially the same concentration of the polymer additive. In one or more embodiments, the concentration of the polymer additive is greater than or equal to about 10% by volume.

Another aspect of the invention is directed to a multi-layered material comprising a first layer comprising a first material that comprises a first thermoplastic elastomer; and a polymer additive substantially uniformly distributed within the first material, the polymer additive having hydrophilic segments and hydrophobic segments, wherein a concentration of the polymer additive in the first material is less than or equal to about 40% by volume and greater than or equal to about 1% by volume. The multi-layered material includes a second layer comprising a second material that comprises a second thermoplastic elastomer.

In one or more embodiments, the first and second materials have different durometers. In one or more embodiments, the first material includes an aqueous and/or solvated therapeutic agent for delivery to a mammalian subject.

Another aspect of the invention is directed to a multi-layered material comprising a first layer that comprises a first thermoplastic elastomer, wherein a plurality of segmented regions are defined in the first thermoplastic elastomer; and a polymer additive substantially uniformly distributed within a plurality of regions of the first thermoplastic elastomer to define additive segments, the polymer additive having hydrophilic segments and hydrophobic segments, wherein a concentration of the polymer additive in each region is less than or equal to about 40% by volume and greater than or equal to about 1% by volume. The multi-layered material includes a second layer comprising a second material that comprises a second thermoplastic elastomer.

In one or more embodiments, the first thermoplastic elastomer is different than the second thermoplastic elastomer.

Another aspect of the invention is directed to a method for manufacturing. The method includes combining a thermoplastic elastomer and a polymer additive to form a material, wherein the polymer additive is substantially uniformly distributed within the material, the polymer additive includes hydrophilic segments and hydrophobic segments, and the material includes a first liquid that includes water and/or a first aqueous solution. The method further includes absorbing at least some of the first liquid into the polymer additive to reduce a tackiness of the compounded material; exposing the compounded material to a second liquid that includes water and/or a second aqueous solution; and absorbing at least some of the second liquid into the polymer additive.

In one or more embodiments, the method further comprises partially hydrating the polymer additive with the first liquid. In one or more embodiments, the method further comprises fully hydrating the polymer additive with the second liquid.

In one or more embodiments, the method further comprises exposing the compounded material to the second aqueous solution, the second aqueous solution including a therapeutic agent. In one or more embodiments, the therapeutic agent comprises a drug.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1A is a block diagram of a material according to an embodiment.

FIG. 1B is a simplified illustration of a single-screw extruder according to an embodiment.

FIG. 2 is a simplified illustration of an example pellet of the material illustrated in FIG. 1A.

FIG. 3 illustrates the chemical structure of an example of a partially hydrolyzed polyacrylonitrile according to an embodiment.

FIG. 4 is an end view of a material that is formed as a tube according to an embodiment.

FIG. 5A is an example cross-sectional view of a therapeutic-delivery device according to an embodiment.

FIG. 5B illustrates a container that includes a reservoir of therapeutic and the therapeutic-delivery device illustrated in FIG. 5A.

FIG. 6A is a cross section of the therapeutic-delivery device illustrated in FIG. 5A according to an embodiment.

FIG. 6B is a cross section of the therapeutic-delivery device illustrated in FIG. 5A according to another embodiment.

FIG. 7 is a cross-sectional view of a multi-layered material according to an embodiment.

FIG. 8 is an end view of an apparatus for melting and/or fusing the multi-layer material illustrated in FIG. 7.

FIG. 9 is a cross-sectional view of the apparatus illustrated in FIG. 8.

FIG. 10 illustrates the multi-layered material illustrated in FIG. 7 where the outer layer is cut to a desired axial length.

FIG. 11 is a flow chart of a method for manufacturing a material according to an embodiment.

### DETAILED DESCRIPTION

A material is formed of a thermoplastic elastomer and a polymer additive. The polymer additive is uniformly and/or substantially uniformly distributed within the material. The polymer additive includes soft hydrophilic segments and hard hydrophobic segments. Water and/or an aqueous solution in the material can be absorbed by the polymer additive, which can reduce the stickiness and/or tackiness of the material. In some embodiments, the polymer additive can also absorb an aqueous and/or solvated therapeutic agent that can be released when the material is placed into a mammal. The material can be delivered in any aqueous environment. For example, the material can be delivered as a marine coating, a water treatment system coating, or a coating implanted in human or animal anatomy.

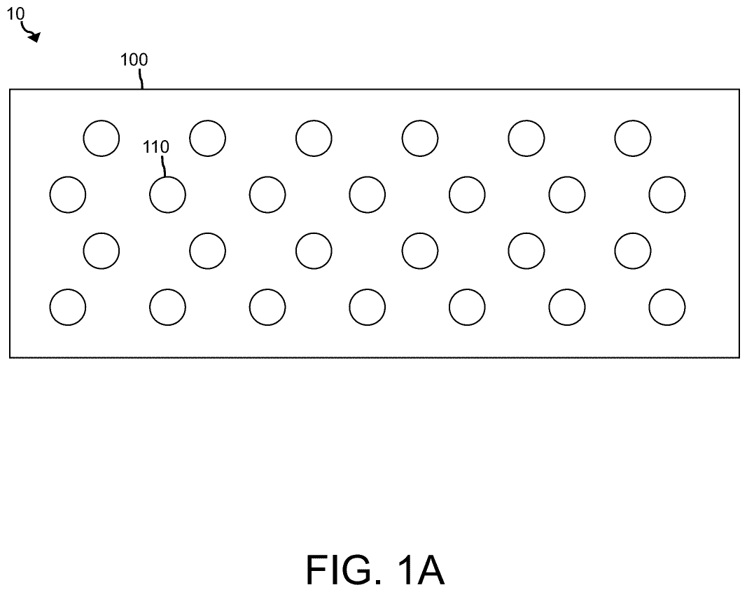

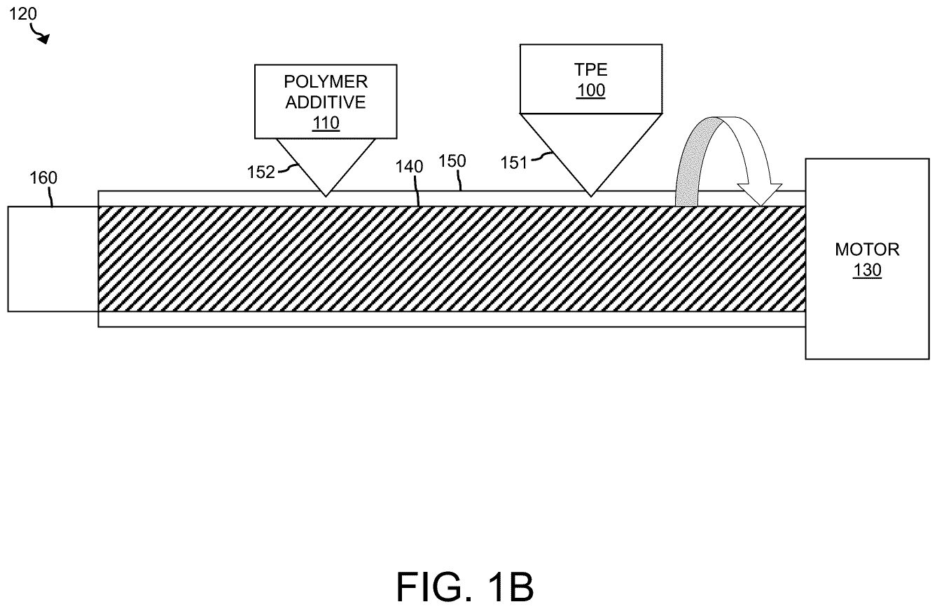

FIG. 1A is a block diagram of a material 10 according to an embodiment. The material 10 is a compound or mixture of a thermoplastic elastomer (“TPE”) 100 and a polymer additive 110. The polymer additive 110 is uniformly and/or substantially uniformly distributed within the material 10. When the polymer additive 110 is substantially uniformly distributed within the material 10, the concentration of the polymer additive 110 can vary by less than about 5% (e.g., about 0% to about 5%) by weight. Additionally or alternatively, when the polymer additive 110 is substantially uniformly distributed within the material 10, the concentration of the polymer additive 110 can vary by less than about 5% (e.g., about 0% to about 5%) by volume within the volume of the material 10. As used herein, “about” means plus or minus 10% of the relevant value.

The TPE 100 can include or consist of one or more thermoplastic polyurethanes (“TPUs”) and/or other TPEs such as a polyether block amide (e.g., PEBAX), a styrene block copolymer (e.g., CFLEX), an olefinic block copolymer (e.g., Percuflex, available from Boston Scientific Corporation), and/or soft silicone. The TPE 100 can have a soft shore durometer, such as between about 25 A and about 85 A.

TPEs can include any thermoplastic elastomer that is converted or further processed using heat to promote a melt flow of the solid phase resin. Two or more resins can be combined and mixed homogenously in a melt flow. The resin(s) are typically introduced at different entrances to the melt flow. A screw or helical shaft can advance the melt flow and mix the combined materials. The polymer additive 110 can be added to the melt flow in solid or liquid (e.g., melted) form and mixed homogenously with the resin(s).

The polymer additive 110 can be partially hydrolyzed. For example, the polymer additive 110 can include soft hydrophilic segments and hard hydrophobic segments. The percentage of soft hydrophilic segments can be determined based on the percent hydrolyzation of the polymer additive. In one example, the polymer additive 110 includes or consists of a partially hydrolyzed polyacrylonitrile (“PH-PAN”).

The material 10 can have a polymer additive concentration (e.g., by weight and/or by volume) of less than or equal to about 40% (and greater than 0%), including about 10%, about 20%, about 30%, and any value or range between any two of the foregoing concentrations. In some embodiments, the polymer additive concentration (e.g., by weight and/or by volume) can be less than about 10% (and greater than 0%), such as about 1%, about 2%, about 4%, about 6%, about 8%, and any value or range between any two of the foregoing concentrations. Thus, in some embodiments, the polymer additive concentration (e.g., by weight and/or by volume) can be in the range of about 1% to about 40%, including any value or sub-range with this range, such as about 1% to about 20% or about 20% to about 40%.

In some embodiments, the material 10 can be initially created in a concentrated form with an initial polymer additive concentration in the range of about 30% by volume up to about 40% by volume (or higher). Additional volume of the same or similar TPE can later be added to the material 10 such that the material has a final polymer additive concentration (e.g., about 1% to about 20% by volume, or higher) that is lower than the initial polymer additive concentration. The material having the initial polymer additive concentration can be divided into different volumes or lots to create different respective final polymer additive concentrations.

The TPE 100, when provided dry, will be sticky and/or tacky which increases the coefficient of friction of the material 10. The TPE 100 can absorb water (or an aqueous solution) that can cause the TPE 100 and the material 10 to absorb water (or the aqueous solution) and reduce the coefficient of friction of the material 10. For example, the polymer additive 110 is configured to absorb some or all of the water (or aqueous solution) from the TPE 100 to reduce the stickiness or tackiness of the TPE 100 and the material 10 and thus reduce the coefficient of friction of the material 10 (e.g., compared to when the material 10 only includes the TPE 100).

For example, a soft durometer (e.g., 85 A) TPU (or another TPE) may absorb between about 5% and about 20% water (by volume) or more. When the TPU is mixed/compounded with PH-PAN (or another polymer additive 110), the PH-PAN absorbs some or all of the water (or aqueous solution) absorbed by the TPU. The transfer of water/aqueous solution from the TPU to the PH-PAN reduces the stickiness and/or tackiness of the TPU surface in dry ambient conditions. Later, when the TPU surface is exposed to water, and/or an aqueous solution, the PH-PAN additive would absorb the aqueous solution and become lubricious, which reduces surface tension and/or friction of the material surface. The polymer additive 110 (e.g., PH-PAN) is homogenously distributed throughout the material 10 including the material's surface, allowing the surface to become lubricious.

The PH-PAN can absorb up to about 600% water (or aqueous solution), by volume. Accordingly, a small percentage concentration (e.g., by weight and/or by volume) of PH-PAN additive (or another polymer additive 110) can be used to absorb moisture in the host TPU to provide a stronger more elastic compounded polymer material 10. A technical advantage of using a high-swell polymer additive 110 in the material 10 is that the polymer additive 110 can be added in low percentage concentrations volumetrically, which can preserve or maintain the material characteristics/properties of the TPE 100 (e.g., host polymer). Additional polymer additive 110 in the TPE 110 may affect the material characteristics/properties, such as its tensile strength and/or elongation.

Compounding the polymer additive 110 with the host TPE 100 resin can be accomplished by melt flowing the host polymer TPE 100 resin with a single or twin screw extruded or metered into the melt flow within the scope of a controlled process.

FIG. 1B is a simplified illustration of a single-screw extruder 120 according to an embodiment. The single-screw extruder 120 can be used to produce the material 10. The single-screw extruder 120 includes a motor 130 that is mechanically coupled to a screw shaft 140. The screw shaft 140 is surrounded by a wall 150. TPE 100 resin(s) are added through a first hopper 151 and polymer additive 110 is added through a second hopper 152. The first hopper 151 is located between the second hopper 152 and the motor 130.

The wall 150 and/or the screw shaft 140 are heated to melt the TPE 100 resin(s) and optionally the polymer additive 110. The concentration (e.g., by weight and/or by volume) of polymer additive 110 in the material 10 can be set by adjusting the relative feed rates of the first and second hoppers 151, 152.

The extruder 120 outputs an extruded rod 160 of the material 10. The rod 160 can be cooled and then chopped or pulverized into pellets, which can be used as raw material to produce a device, such as catheter tube.

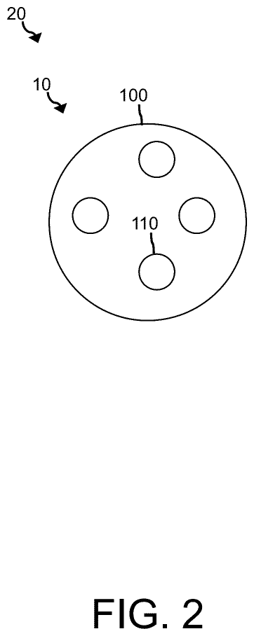

The corresponding ratio of polymer additive 110 metered into the TPE 100 melt flow will be homogenously a consistent ratio and when chopped or pelletized each pellet will contain a corresponding same ratio of polymer additive 110 and host TPE 100 resin. Metering of a high-melt-flow additive, such as PH-PAN, which remains homogenous in pellet form is a valuable attribute. An example pellet 20 of the material 10 is illustrated in FIG. 2.

The polymer additive 110 can hydrodynamically decrease friction in the melt flow of the TPE 100 due to the polymer additive 110 being homogenously and/or substantially homogenously dispersed within the host TPE 100. The reduced friction can reduce boundary-layer issues and can provide, for example, higher flow rates at lower pressures, reduced drag and turbulence, and a corresponding reduced Reynolds number.

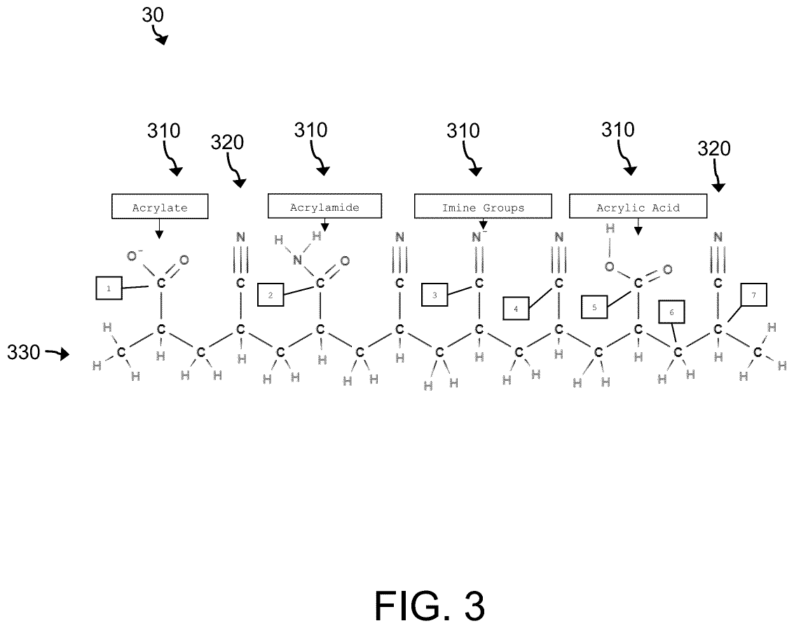

An example of a PH-PAN 30 with soft hydrophilic segments 310 and hard hydrophobic segments 320 is illustrated in FIG. 3. The soft hydrophilic segments 310 can comprise or consist of acrylate, acrylamide, imine, and/or acrylic acid groups formed on a PAN backbone 330. The hard hydrophobic segments 320 can comprise or consist of nitrile groups, which have not been converted to soft hydrophilic segments 310 during partial hydrolyzation.

The ratio of soft hydrophilic segments 310 to hard hydrophobic segments 320 can determine the percentage swell of the PH-PAN 30 when exposed to and/or saturated with an aqueous solution, which corresponds to (and enhances) the amount of aqueous solution absorbed and/or saturated by the PH-PAN 30. The soft hydrophilic segments 310 can be uniformly and/or substantially uniformly distributed within (or dispersed throughout) the PH-PAN backbone 330.

The PH-PAN 30 can have a hydrolyzation percentage of about 30% to about 50% (i.e., about 30% to about 50% of the segments are soft hydrophilic segments and the balance (e.g., 70% to 50%) of the segments are hydrophobic crystalline segments and/or other derivatives due to hydrolyzation of the PH-PAN), including about 35%, about 40%, about 45%, and any value or range between any two of the foregoing percentages.

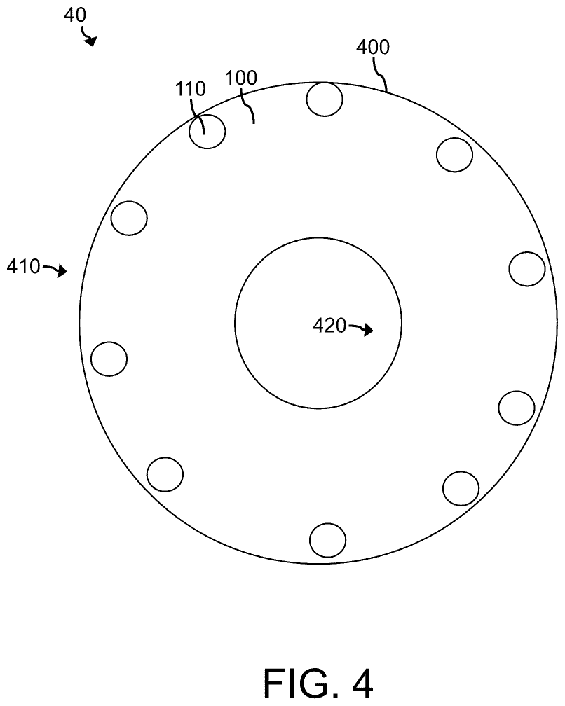

FIG. 4 is an end view of a material 40 that is formed as a tube 400 according to an embodiment. The material 40 can be the same as or different than material 10. The tube 400 can be in the form of a stent, a catheter, a condom, or in another form (e.g., like a flat sheet). In material 40, the polymer additive 110, which was previously distributed homogenously (e.g., as illustrated in FIG. 1), has settled on the outer surface 410 of the material 40 and of the tube 400, according to an embodiment. The material 40 can be formed by mixing the TPE 100 and the polymer additive 110 when the TPE 100 and optionally the polymer additive 110 is/are solvated. When both the TPE 100 and the polymer additive 110 are solvated, the solvents should be compatible.

In some embodiments, the material 40 can be used or applied when the polymer additive 110 is settled on the outer surface 410 of the material 40. For example, the material 40 can be configured such that one surface (e.g., the outer surface 410) is lubricious and/or detackified (e.g., has a relatively low coefficient of friction) while the opposing surface (e.g., the inner surface 420) remains sticky or tacky (e.g., has a relatively high coefficient of friction).

In other embodiments, the material 40 can be mixed prior to use/application so that the polymer additive 110 is substantially uniformly distributed within the material 40.

It is believed that the settling can be caused by relative densities, particle size, lack of affinity for solvent/solubility, and/or other properties of the compound. Looking at common solvents for the polymer additive 110 and the TPE 100, settling may be an issue if the polymer additive 110 is totally solvated. However, different TPEs may require different solvents for solvation so it is reasonable to suggest solution grades (e.g., the TPE resin can have no other additive used as an aide for molding or extrusion such as a wax or lubricant) will have settling and as such they may need to be mixed before use to prevent settling. For example, the polymer additive 110 may settle when mixed with a solvated resin formulation of TPE 100 and left in a container for storage. Mixing (e.g., shaking, stirring, and/or agitating) the container before use (e.g., to form an extruded product) can address any settling that may have occurred. In addition, the percent concentration (e.g., by weight and/or by volume) of TPE 100 in the material 10 can be varied to provide different corresponding viscosities.

TPEs can be solvated by polar solvents. Some of these polar solvents may solvate the polymer additive in addition to the TPE. For example, TPU and PH-PAN can be solvated using dimethyl sulfoxide (DMSO). However, while PH-PAN will go into solution relatively quickly, TPU may not. Similarly, tetrahydrofuran (THF) will put TPU into solution but not PH-PAN. Accordingly, the compound's formulation should be considered when determining which manufacturing process which may be beneficial for the end product. For example, a TPU/PH-PAN additive compound in a THF solvent will exhibit a dispersion of the polymer additive which will eventually settle and therefore require vigorous mixing before use which may be minimized by higher concentrations of TPU. Conversely, a solvent which solvates both TPE and additive will provide solution maintaining a uniform mixture less likely to exhibit settling.

The solution can be used for dipping/coating a resin or for thermoplastic processing (e.g., extrusion/coating) or for thermoplastic processing (extrusion/injection molding).

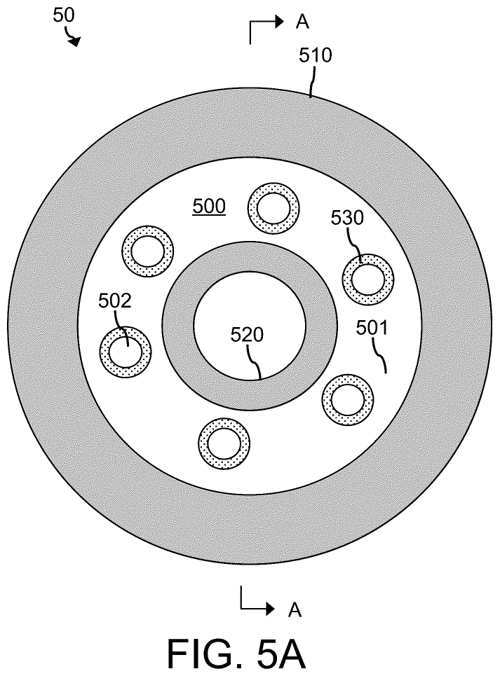

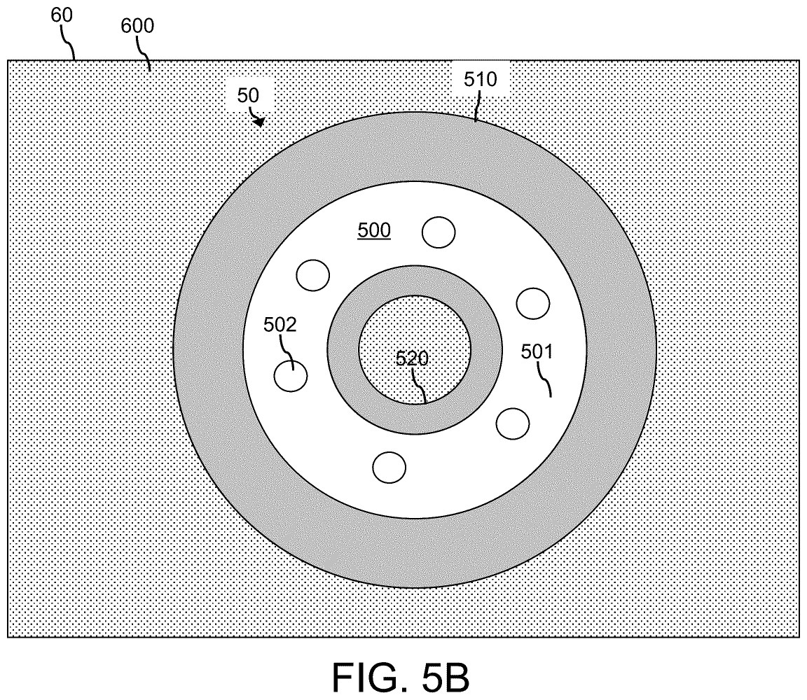

FIG. 5A is an example cross-sectional view of a therapeutic-delivery device 50 according to an embodiment. The therapeutic-delivery device 50 includes a therapeutic-delivery layer 500, an optional outer layer 510, and an optional inner layer 520. When the therapeutic-delivery device 50 includes the optional outer layer 510 and/or the optional inner layer 520, the therapeutic-delivery device 50 is formed of a multi-layered material. The therapeutic-delivery device 50 is shown as a tube but can be in the form of a stent, a catheter, or other shapes or forms, as will be appreciated by those of skill in the art.

The therapeutic-delivery layer 500 is formed of a material that includes a TPE 501 and a polymer additive 502 that is uniformly and/or substantially uniformly distributed within the material 502 and the TPE 501. The TPE 501 can be the same as or different than TPE 100. The polymer additive 502 can be the same as or different than polymer additive 110. The therapeutic-delivery layer 500 can be formed of the same material or of a different material than material 10 and/or material 40.

The polymer additive 502 can absorb and release a therapeutic agent 530, such as a drug, a medicine, or another therapeutic agent, which can be in aqueous form. For example, the therapeutic-delivery device 50 can first be placed into a container 60 that includes a reservoir 600 of therapeutic agent 530, for example as illustrated in FIG. 5B. When submersed in the reservoir 600, the therapeutic agent 530 is transported through the therapeutic-delivery layer 500 and the optional outer and/or inner layers 510, 520 as the therapeutic agent 530 seeks equilibrium between the reservoir 600 and the therapeutic-delivery device 50. The therapeutic agent 530 transported through the therapeutic-delivery layer 500 is absorbed by the polymer additive 502. After sufficient therapeutic agent 530 is absorbed by the polymer additive 502, the therapeutic-delivery device 50 can be placed into a mammal (e.g., human) where the polymer additive 502 releases the therapeutic agent 530 into the mammal as equilibrium is reached between the mammal and the therapeutic-delivery device 50 for the therapeutic agent 530.

The delivery rate of the therapeutic agent 530 (e.g., the rate at which equilibrium is reached) can be controlled by the Shore durometer of the therapeutic-delivery layer 500, the thickness of the therapeutic-delivery layer 500, and/or the concentration of the therapeutic agent 530. In general, a lower Shore durometer (softer material) increases the therapeutic agent deliver rate (and corresponding diffusion rate) compared to a higher Shore durometer (harder material), although molecular weights of polymer and solute need to be considered. In addition, a material having a lower Shore durometer usually can absorb aqueous media quicker and in greater amounts compared to a material having a higher Shore durometer.

In some embodiments, the delivery rate of the therapeutic agent 530 can be controlled by including the optional outer layer 510 and/or the optional inner layer 520. For example, including both outer and inner layers 510, 520 increases the effective thickness of the therapeutic-delivery layer 500, which can reduce the delivery rate of the therapeutic agent 530. The outer and/or inner layers 510, 520 can include a TPE that can be the same or different than TPE 501. The TPE in the outer layer 510 can be the same or different than the TPE in the inner layer 520.

In one example, the inner layer 520 can include a TPE having a relatively low Shore durometer and the outer layer 510 can include a TPE having a relatively high Shore durometer. The relative difference in Shore durometers between the outer and inner layers 510, 520 can direct the therapeutic-delivery potential inwards such that more therapeutic agent 530 is delivered inwardly, from the therapeutic-delivery layer 500 through the inner layer 520, than outwardly, from the therapeutic-delivery layer 500 through the outer layer 510. In another example, the inner layer 520 can include a TPE having a relatively high Shore durometer and the outer layer 510 can include a TPE having a relatively low Shore durometer. The relative difference in Shore durometers between the outer and inner layers 510, 520 can direct the therapeutic-delivery potential outwards such that more therapeutic agent 530 is delivered outwardly, from the therapeutic-delivery layer 500 through the outer layer 510 than inwardly, from the therapeutic-delivery layer 500 through the inner layer 520.

It is noted that the therapeutic-delivery device 50 can be used to deliver substances other than (or in addition to) the therapeutic agent 530. Additionally or alternatively, the therapeutic-delivery device 50 can be used to remove or extract a substance from a target location (e.g., when the therapeutic-delivery device 50 is placed without a substance, such as the therapeutic agent 530, absorbed by the polymer additive 502).

When the therapeutic-delivery device 50 only includes the therapeutic-delivery layer 500, the therapeutic-delivery device 50 can be formed by extrusion or injection molding. When the therapeutic-delivery device 50 includes the therapeutic-delivery layer 500 and the outer layer 510 and/or the inner layer 520, the therapeutic-delivery device 50 can be formed by co-extrusion, overcoat processing (e.g., dip coating), and/or injection molding. Other manufacturing techniques can be used, as will be appreciated by those of skill in the art.

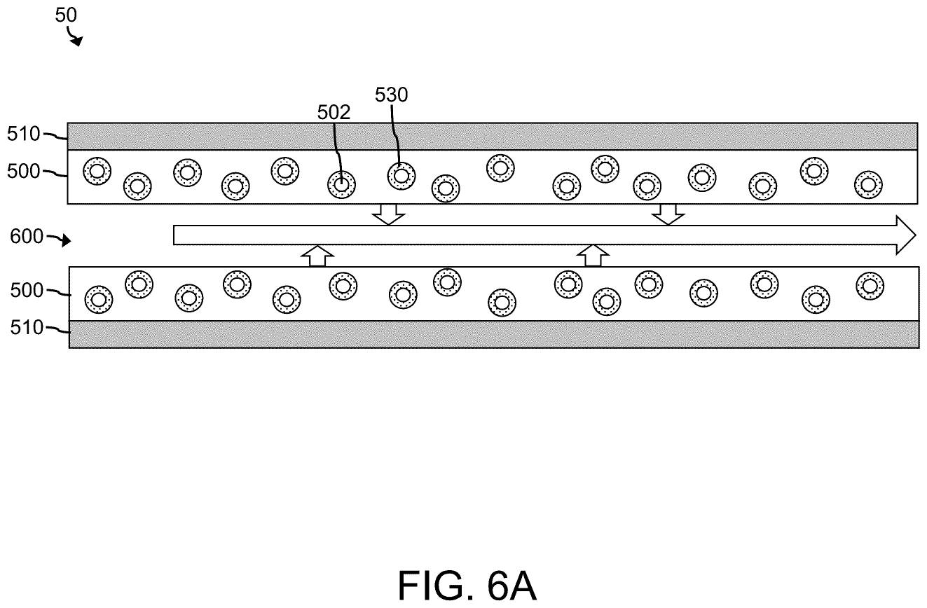

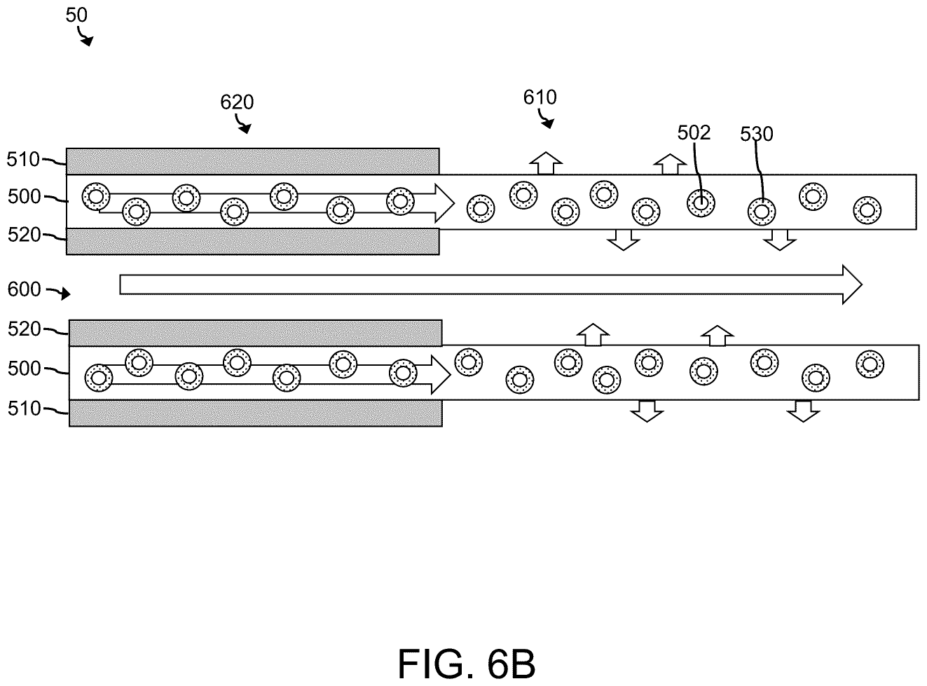

FIG. 6A is a cross section through section A of the therapeutic-delivery device 50 illustrated in FIG. 5A according to an embodiment. In the illustrated embodiment, the therapeutic-delivery device 50 includes the therapeutic-delivery layer 500 and the outer layer 510 but not the optional inner layer 520. A channel 600 is formed in the middle of the therapeutic-delivery device 50 through which fluid, such as bodily fluid, can flow when the therapeutic-delivery device 50 is implanted in a mammal. The therapeutic agent 530 diffuses inwardly through the therapeutic-delivery layer 500 and into the channel 600 where it is then delivered to the mammal at a substantially uniform rate. Diffusion of the therapeutic agent 530 through the therapeutic-delivery layer 500 occurs more rapidly than through the outer layer 510 (e.g., due to the difference in their respective Shore durometers), and thus the therapeutic agent 530 follows the path of least resistance.

FIG. 6B is a cross section through section A of the therapeutic-delivery device 50 illustrated in FIG. 5A according to another embodiment. In the illustrated embodiment, the therapeutic-delivery device 50 includes the therapeutic-delivery layer 500, the outer layer 510, and the inner layer 520. The outer and inner layers 510, 520 are shortened compared to the therapeutic-delivery layer 500 to define an exposed portion 610 and an unexposed portion 620 of the therapeutic-delivery layer 500. The outer and inner layers 510, 520 are terminated and do not surround the therapeutic-delivery layer 500 in the exposed portion 610. The outer and inner layers 510, 520 surround the therapeutic-delivery layer 500 in the unexposed portion 620.

The therapeutic agent 530 diffuses laterally through the therapeutic-delivery layer 500 in the unexposed portion 620 and into the exposed portion 610. In the exposed portion 610, the therapeutic agent 530 diffuses radially (e.g., inwardly towards the channel 600 and outwardly away from the channel 600). The therapeutic-delivery layer 500 that diffuses into the channel 600 is then delivered to the mammal at a substantially uniform rate. Diffusion of the therapeutic agent 530 through the therapeutic-delivery layer 500 occurs more rapidly than through the outer and inner layers 510, 520 (e.g., due to the difference in their respective Shore durometers), and thus the therapeutic agent 530 follows the path of least resistance.

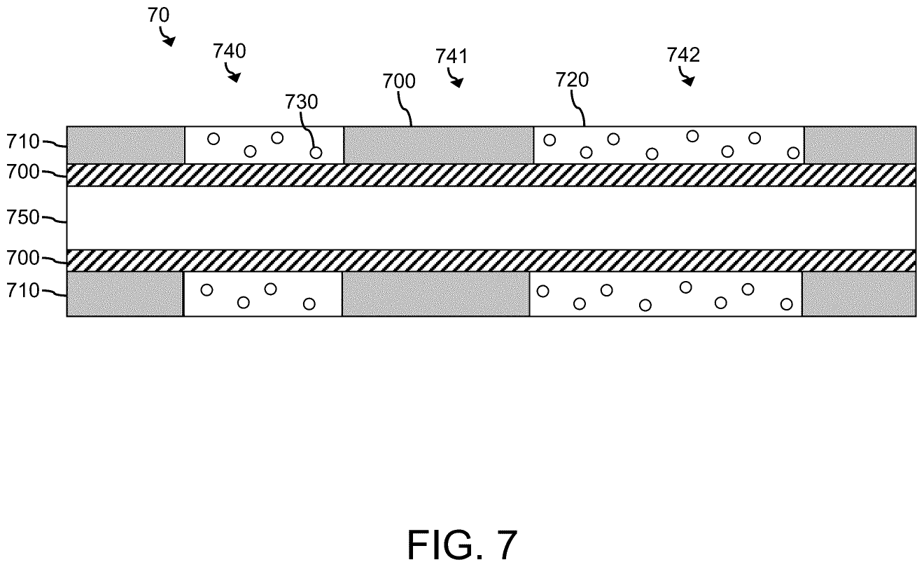

FIG. 7 is a cross-sectional view of a multi-layered material 70 according to an embodiment. The multi-layered material 70 includes an inner TPE layer 700 and an outer TPE layer 710. The inner and outer TPE layers 700, 710 are in direct physical contact with each other. The inner layer 700 is optional and can be omitted in some embodiments. Alternatively, the inner and outer TPE layers 700, 710 can be switched.

The inner TPE layer 700 includes or consists of a TPE. The outer TPE layer 710 includes one or more regions 720 in which a polymer additive 730 is uniformly distributed and/or substantially uniformly distributed with the respective region(s) 720. The region(s) 720 form one or more segments 740 including unfilled segments 741 and filled or additive segments 742. The polymer additive 730 is included in the filled/additive segments 742. The multi-layered material 70 can include additional or fewer regions 720 and segments 740.

The inner layer 700 can be formed by cutting unfilled segments 741 and filled/additive segments 742 from respective tubes of the respective materials and placing them in an alternating pattern coaxially over an inner tube layer or a sheath. The neighboring segments 741, 742 can be bonded by applying heat and/or an adhesive.

The TPE in the inner and outer TPE layers 700, 710 can be the same as or different than TPE 100 and/or 501. The TPE in the inner TPE layer 700 can be the same as or different than the TPE in the outer TPE layer 710. The polymer additive 730 can be the same as or different than polymer additive 110 and/or 502. Each region 740 can have the same concentration or a different concentration of polymer additive 740.

The multi-layered material 70 can be formed as a tube or in another shape. The multi-layered material 70 can be formed on a mandrel and/or can have a hollow center 750.

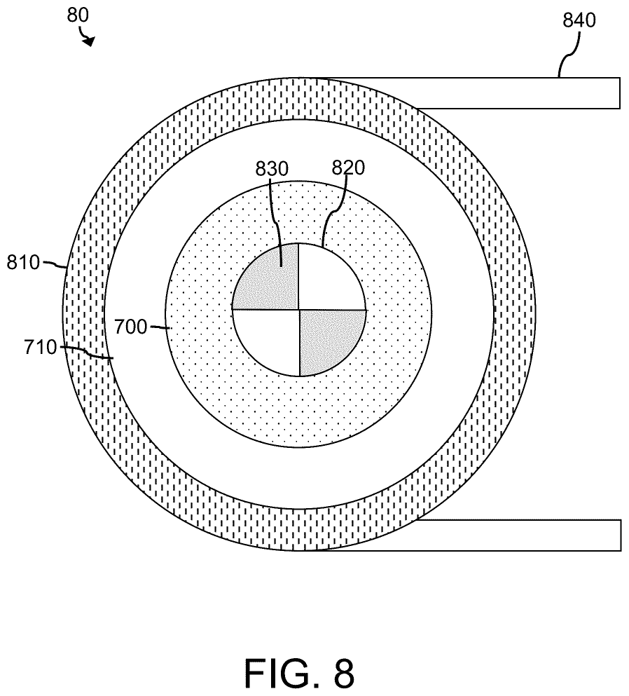

FIG. 8 is an end view of an apparatus 80 for melting and/or fusing the multi-layer material 70. The apparatus 80 includes one or more inductive coils 810 that produce RF energy that can heat a mandrel 820, which is configured to conduct RF energy. Heat is transferred outwardly from the mandrel 820 to the inner and outer TPE layers 700, 710 and causes the inner and outer TPE layers 700, 710 to melt and/or fuse together. One or more channels 830 can be defined in the mandrel 820 through which water or another liquid can flow to cool the mandrel 820. One or more cooling lines 840 is/are fluidly coupled to the inductive coil(s) 810 and to the mandrel 820.

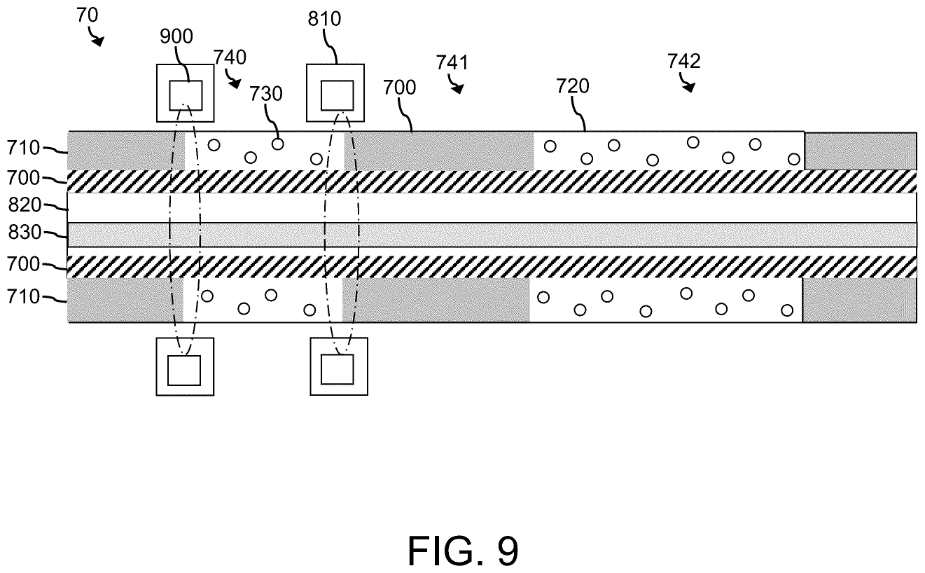

FIG. 9 is a cross-section through section A in FIG. 8. The cross section reveals that water/liquid cooling channels 900 are defined in the inductive coils 810. The water/liquid cooling channels 900 can cool the inductive coils 810, such as after RF energy is produced.

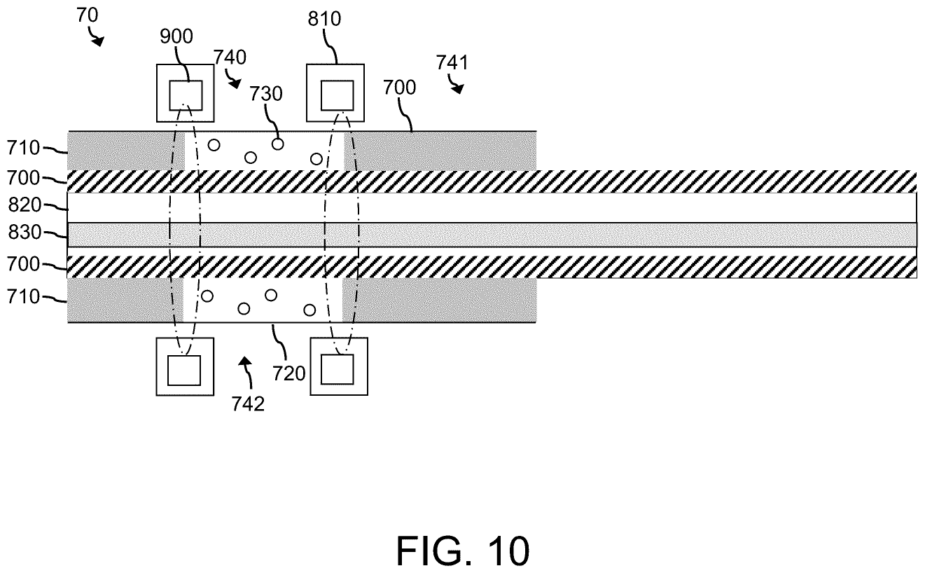

In some embodiments, the outer TPE layer 710 can be cut to a desired axial length, as illustrated in FIG. 10.

Mandrel material/design, solvent, and polymer additive concentration should be considered when dipping to form a profile and/or coating a specific material. For example, an aluminum mandrel may attract a PH-PAN polymer additive inwardly when dipped vertically in such a manner that the inside surface (in contact with/adjacent to the mandrel) exhibits lower surface energy than the outer surface. In that manner the tackiness of the outer surface is reduced, while the inner surface is very lubricous. This approach may be advantageous when forming balloons, condoms, and/or gloves, for example.

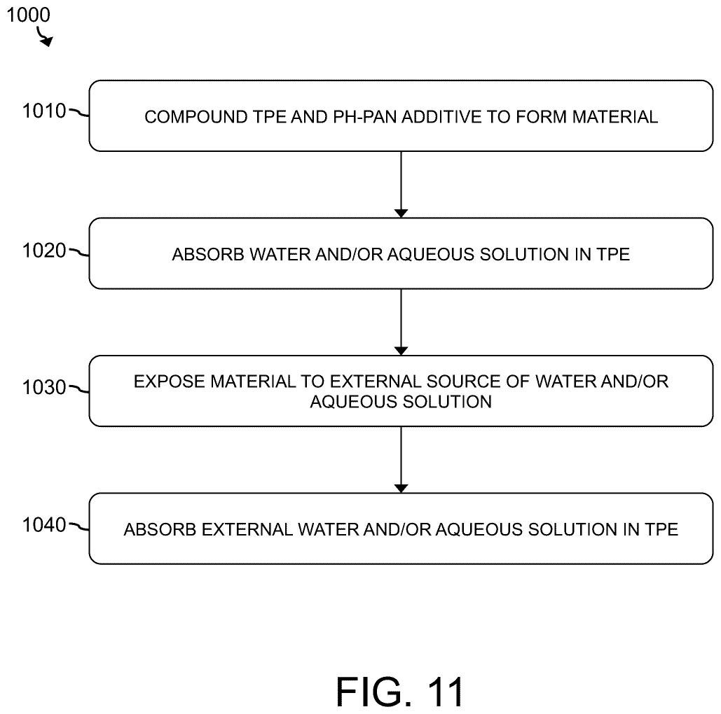

FIG. 11 is a flow chart of a method 1000 for manufacturing a material according to an embodiment. In step 1010, TPE and a polymer additive are mixed/compounded to form the material. The TPE can be a TPU. The polymer additive can be PH-PAN. The PH-PAN additive can have a percentage hydrolyzation to produce a target/desired effect in the material. In addition, the concentration (e.g., by weight and/or by volume) of the PH-PAN additive in the TPE can be selected to produce a target/desired effect in the material. The material can be formed through extrusion (e.g., melt flowing the host polymer TPE resin), co-extrusion, injection molding, and/or dipping. Co-extrusion, injection molding, and/or dipping can be used to form a multi-layered material.

In step 1020, the polymer additive absorbs at least some of the water and/or aqueous solution in the corresponding TPE, which reduces the tackiness and/or stickiness of the material. The polymer additive can become partially hydrated in this step.

In step 1030, the material is exposed to a liquid such as an external source of water and/or an aqueous solution. For example, the material can be immersed in a container that holds the water and/or aqueous solution. The aqueous solution can optionally include a therapeutic agent (e.g., therapeutic agent 530).

In step 1040, the polymer additive absorbs the water and/or aqueous solution, which increases the lubricity of the material. When the aqueous solution includes a therapeutic agent, the material can be used as a therapeutic-delivery device. The polymer additive can become fully hydrated in this step. The therapeutic-delivery device can then be placed in a target location, such as in a cavity in a mammal, to release the absorbed therapeutic agent.

In another embodiment, the compound can be formed into a paint. The paint can be water-based or solvent-based and includes a dispersion of the polymer additive. The paint can be used as an anti-graffiti coating that can be applied to a building or other structure and will resist absorbing inks and related media that otherwise would stain and/or be absorbed by conventional paints and coatings. And even if absorbed, the stain is likely to be water-soluble or water-based and, in that manner, easily washed away.

The paint could also be used as a marine or naval antifouling coating the product would be environmentally friendly and adherence simply accomplished.

In these applications, particle size of the polymer additive in the paint may contribute to the dispersion not settling and as such is preferably about 100 microns in average and/or median size. The polymer additive can be solvated in a base material (e.g., TPE) appropriately as to manage whether or not the polymer additive might settle in the solvated host polymer solution. Paint can be considered a coating that is typically a lower-viscosity liquid with a corresponding lower percent concentration (e.g., by weight and/or by volume) of host polymer solution. The lower percent concentration of solvated host polymer has a corresponding lower concentration (e.g., by weight and/or by volume) of polymer additive that remains dispersed in the solvated host polymer solution either because particle size (e.g., surface area) maintains a sense of buoyancy, the ratio/concentration of solid phase additive is low enough to remain dispersed, and/or the polymer additive is indeed solvated and mixed with solvated host polymer. Any settling can be of the polymer additive can be addressed by shaking and/or mixing well before applying the paint.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A material comprising:
a thermoplastic elastomer; and
a polymer additive substantially uniformly distributed within the material, the polymer additive having hydrophilic segments and hydrophobic segments,
wherein a concentration of the polymer additive in the material is less than or equal to about 40% by volume and greater than or equal to about 1% by volume.

2. The material of claim 1, wherein the thermoplastic elastomer comprises a thermoplastic polyurethane.

3. The material of claim 1, wherein the polymer additive comprises a partially hydrolyzed polyacrylonitrile.

4. The material of claim 3, wherein the partially hydrolyzed polyacrylonitrile has a hydrolysis percentage of greater than equal to about 30% and less than or equal to about 50%.

5. The material of claim 1, wherein the material comprises a plurality of pellets, each pellet having substantially the same concentration of the polymer additive.

6. The material of claim 1, wherein the concentration of the polymer additive is greater than or equal to about 10% by volume.

7. A multi-layered material comprising:
a first layer comprising a first material, the first material comprising:
a first thermoplastic elastomer; and
a polymer additive substantially uniformly distributed within the first material, the polymer additive having hydrophilic segments and hydrophobic segments,
wherein a concentration of the polymer additive in the first material is less than or equal to about 40% by volume and greater than or equal to about 1% by volume; and
a second layer comprising a second material that comprises a second thermoplastic elastomer.

8. The multi-layered material of claim 7, wherein the first and second materials have different durometers.

9. The multi-layered material of claim 7, wherein the first material includes an aqueous therapeutic agent that is absorbed by the polymer additive for delivery to a mammalian subject.

10. A multi-layered material comprising:
a first layer comprising:
a first thermoplastic elastomer, wherein a plurality of segmented regions are defined in the first thermoplastic elastomer; and
a polymer additive substantially uniformly distributed within a plurality of regions of the first thermoplastic elastomer to define additive segments, the polymer additive having hydrophilic segments and hydrophobic segments,
wherein a concentration of the polymer additive in each region is less than or equal to about 40% by volume and greater than or equal to about 1% by volume; and
a second layer comprising a second material that comprises a second thermoplastic elastomer.

11. The multi-layered material of claim 10, wherein the first thermoplastic elastomer is different than the second thermoplastic elastomer.

12. A method for manufacturing, comprising the ordered steps of:
combining a thermoplastic elastomer and a polymer additive to form a material, wherein:
the polymer additive is substantially uniformly distributed within the material,
the polymer additive includes hydrophilic segments and hydrophobic segments, and
the material includes a first liquid that includes water and/or a first aqueous solution;
absorbing at least some of the first liquid into the polymer additive to reduce a tackiness of the compounded material;
exposing the compounded material to a second liquid that includes water and/or a second aqueous solution; and
absorbing at least some of the second liquid into the polymer additive.

13. The method of claim 12, further comprising partially hydrating the polymer additive with the first liquid.

14. The method of claim 13, further comprising fully hydrating the polymer additive with the second liquid.

15. The method of claim 12, further comprising exposing the compounded material to the second aqueous solution, the second aqueous solution including a therapeutic agent.

16. The method of claim 15, wherein the therapeutic agent comprises a drug.
